(19) United States
(12) Patent Application Publication
(10) Pub. No.: US 2024/0043406 A1
(43) Pub. Date: Feb. 8, 2024

(54) METHOD FOR SEQUENTIAL ONE-POT SYNTHESIS OF TKX-50

(71) Applicant: EURENCO, Sorgues (FR)

(72) Inventors: Arthur DELAGE, Mormoiron (FR); Geneviève ECK, Monteux (FR); Thibaud ALAIME, Avignon (FR); Frédérick LACEMON, Bagnols sur Ceze (FR); Thomas KLAPOTKE, Munich (DE); Jorg STIERSTORFER, Worthsee (DE); Marc BOLTER, Munich (DE)

(21) Appl. No.: 18/268,474

(22) PCT Filed: Nov. 26, 2021
(86) PCT No.: PCT/FR2021/052105

§ 371 Date: Jun. 20, 2023

(30) Foreign Application Priority Data
Dec. 18, 2020 (FR) FR2013614

(51) Int. Cl. C07D 403/04

(52) U.S. Cl. CPC C07D 403/04

(57) ABSTRACT

The present invention relates to a sequential one-pot synthesis of TKX-50, suitable for larger-scale production, during which an acetyl halide is used during the cyclization of diazidoglyoxime so as to obtain 1,1′-diacetyl-5,5′-bistetrazole, which is then hydrolyzed to 5,5′-bistetrazole-1,1′-diolate, to which compound a hydroxylammonium salt is subsequently added.

### TECHNICAL FIELD

The present invention relates to a method for sequential one-pot synthesis of TKX-50 (or dihydroxylammonium 5-5′-bistetrazole-1,1′-diolate) which advantageously allows larger-scale production of this molecule compared to known methods.

### PRIOR ART

TKX-50 is a promising energetic molecule that has a higher detonation velocity than octogen (HMX) and a sensitivity to various possible aggressions during the life cycle equivalent to that of hexogen (RDX).

A known synthetic route for TKX-50 involves the chlorination of glyoxime into dichloroglyoxime by dichlor (Cl₂). The dichloroglyoxime obtained is then isolated then by reaction with sodium azide (NaN₃) provides diazidoglyoxime, which then reacts with gaseous hydrochloric acid (HCl) in diethyl ether (Et₂O) so as to obtain, after cyclization, a bistetrazole. TKX-50 is obtained after adding the hydroxylammonium salt to the reaction medium.

This synthesis is suitable for a laboratory scale for the manufacture of small amounts of TKX-50 but its use remains more delicate if the synthesis is desired on a larger scale, due to the use of gaseous hydrochloric acid and dichlor which are corrosive gases. It is also desirable to dispense with the use of flammable solvents such as diethyl ether. The publication Golenko and al. «Optimization Studies on Synthesis of TKX-50”, Chinese Journal of Chemistry, 2017, 35, 98-102 is also known.

### DISCLOSURE OF THE INVENTION

The invention relates to a method for sequential one-pot synthesis of TKX-50, comprising at least:
an azidization of a dihalogenoglyoxime or of the diaminoglyoxime so as to obtain the diazidoglyoxime,
a cyclization by reaction of the diazidoglyoxime obtained with an acetyl halide so as to obtain 1,1′-diacetyl-5,5′-bistetrazole,
a hydrolysis of the 1,1′-diacetyl-5,5′-bistetrazole obtained into 5,5′-bistetrazole-1,1′-diolate, and
an ion exchange by adding a hydroxylammonium salt to the 5,5′-bistetrazole-1,1′-diolate obtained so as to obtain TKX-50.

The invention proposes the use of an acetyl halide as a reagent during the cyclization reaction in order to form a new reaction intermediate: 1,1′-diacetyl-5,5′-bistetrazole (designated in the following by “Ac₂BTO”). The proposed synthetic route allows a sequential one-pot synthesis for at least the four steps of azidization, cyclization, hydrolysis and ion exchange while avoiding the use of gaseous hydrochloric acid, unlike the prior art. Thus, no step of isolation of the diazidoglyoxime reaction intermediates, Ac₂BTO and 5,5′-bistetrazole-1,1′-diolate (hereinafter referred to as “BTO”) is necessary and the synthesis is made safer and compatible with an increase in scale, in order to prepare, for example, at least several kilos of TKX-50 in the same reactor.

In an exemplary embodiment, a temperature greater than or equal to 30° C. and less than the boiling temperature of the acetyl halide is imposed during the cyclization.

Such a characteristic further simplifies the synthesis of TKX-50 on a larger scale by imposing a sufficient temperature to avoid any risk of solidification of the reaction medium during the cyclization, while limiting the temperature so as not to bring the acetyl halide to a boil.

The temperature imposed during the cyclization can for example be comprised between 30° C. and 51° C.

According to a particular example, the acetyl halide is acetyl chloride.

According to a particular example, the acetyl halide can be added in a proportion of 2 to 3 equivalents with respect to the diazidoglyoxime to carry out the cyclization.

In a non-limiting exemplary embodiment, the azidization, the cyclization, the hydrolysis and the ion exchange are carried out in a common solvent comprising at least one C₁ to C₄ alcohol, dimethylformamide, acetone or acetonitrile.

These solvents are non-limiting examples allowing sequential one-pot synthesis and their use advantageously has a significantly reduced risk compared to the use of diethyl ether used in the prior art due to their low flammability and these solvents further allow to solubilize the sensitive intermediates of the synthesis in particular diazidoglyoxime and Ac₂BTO which have risks of violent decomposition.

In particular, the common solvent can comprise dimethylformamide.

The choice of dimethylformamide (designated hereinafter by “DMF”) advantageously leads to a high yield of formation of TKX-50, for example greater than or equal to 80% over the entire synthesis.

In an exemplary embodiment, the diazidoglyoxime is obtained by azidization of a dihalogenoglyoxime.

According to a particular example, the dihalogenoglyoxime can be dichloroglyoxime (C₂H₂Cl₂N₂O₂) or dibromoglyoxime (C₂H₂Br₂N₂O₂). In particular, the dihalogenoglyoxime can be dichloroglyoxime.

In particular, the dihalogenoglyoxime can be dichloroglyoxime, and the method can further comprise, prior to the azidization, the formation of the dichloroglyoxime by chlorination of the glyoxime by reaction with N-chlorosuccinimide.

The use of N-chlorosuccinimide (designated in the following by “NCS”) is advantageous because it allows to carry out the chlorination by dispensing with the use of dichlor used in the prior art, thus further improving the safety of the synthesis.

According to one example, a temperature comprised between 30° C. and 80° C. is imposed during the chlorination.

Such a characteristic is advantageous in order to limit the exothermicity of the reaction. Indeed, when the reaction is carried out at ambient temperature, a significant exotherm is observed which can lead to the boiling of the reaction medium, which increases the risk of safety of the method.

Chlorination as well as azidization, cyclization, hydrolysis, and ion exchange can be performed in dimethylformamide.

DMF has the additional advantage of being a solvent for dissolving the NCS, which allows to carry out the sequential one-pot synthesis from the chlorination of glyoxime so as to obtaining TKX-50.

According to one variant, the diazidoglyoxime is obtained by azidization of the diaminoglyoxime.

The invention also relates to a method for manufacturing an energy composition, comprising at least:
implementing a method as described above so as to obtain TKX-50, and
obtaining the energy composition from the TKX-50 thus obtained.

The energetic composition can be an explosive composition or a propellant composition.

### BRIEF DESCRIPTION OF THE DRAWINGS

FIG. 1 is an overall reaction diagram of an example of the synthesis of TKX-50 according to the invention.

### DESCRIPTION OF EMBODIMENTS

Azidization causes a dihalogenoglyoxime or diaminoglyoxime to react with an azide ion of formula N₃⁻ or another azidizing agent. The general chemical structure of a dihalogenoglyoxime (C₂H₂X₂N₂O₂) is given below, in which X denotes a halogen atom, which can be chlorine or bromine. In particular, dichloroglyoxime is used (X═Cl).

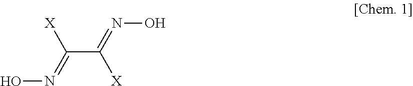

Diaminoglyoxime (C₂H₆N₄O₂) has the chemical structure shown below.

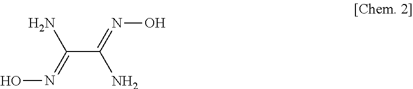

Azidization is a reaction known per se. It can be carried out by reacting dihalogenoglyoxime or diaminoglyoxime with sodium azide (NaN₃) or another azidizing agent. A temperature comprised between 0° C. and 20° C., for example between 0° C. and 10° C., can be imposed during the azidization. This temperature can be imposed for a duration comprised between 40 minutes and 120 minutes.

During azidization, there is substitution of the halogen X or of the amino group —NH₂ by the azide. Following azidization, diazidoglyoxime (C₂H₂N₈O₂) is obtained, the chemical structure of which is illustrated below.

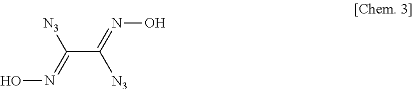 [SMILES: [N-]=[N+]=NC(=N\O)/C(N=[N+]=[N-])=N/O]

The yield of the formation of diazidoglyoxime from dihalogenoglyoxime or diaminoglyoxime can be greater than or equal to 90%.

The method continues with the cyclization reaction during which the diazidoglyoxime obtained is contacted with an acetyl halide (AcX where X designates a halogen atom) so as to obtain Ac₂BTO. The chemical structure of acetyl halide is provided below, X can be chlorine or bromine. In particular, acetyl chloride can be used (X═Cl).

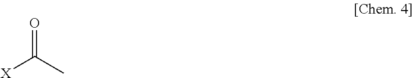

The chemical structure of the Ac₂BTO obtained is provided below.

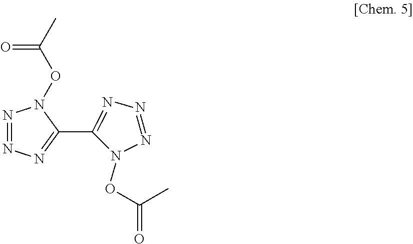 [SMILES: CC(=O)On1nnnc1-c1nnnn1OC(C)=O]

The cyclization results in the formation of a 5,5′-bistetrazole structure with, furthermore, nucleophilic substitution of the hydroxyl groups (—OH) of the oxime functions of the diazidoglyoxime on the acetyl with departure of the halogen so as to obtain Ac₂BTO.

The temperature of the reaction medium can be increased to a temperature comprised between 30° C. and the boiling point of the acetyl halide before the addition of the acetyl halide, for example to a temperature comprised between 30° C. and 51° C. The acetyl halide is subsequently added to the diazidoglyoxime at this temperature. The temperature imposed during the cyclization can be less than the boiling point of the acetyl halide, for example less than or equal to 51° C., or even comprised between 30° C. and 51° C.

This temperature can be imposed for a duration comprised between 5 hours and 15 hours. According to one example, the reaction mixture can be free of alcohol during the cyclization. As indicated above, the reaction medium is devoid of gaseous hydrochloric acid during the cyclization in particular.

The yield of the formation of Ac₂BTO from diazidoglyoxime can be greater than or equal to 90%.

The method continues with a hydrolysis of Ac₂BTO into BTO. This hydrolysis can be carried out by adding ice and/or liquid water to the Ac₂BTO. After hydrolysis, a solution comprising the BTO is obtained.

The method continues by adding the hydroxylammonium salt, for example a hydroxylammonium halide, to the BTO. The salt has the formula NH₃OH⁺Y⁻, where Y denotes for example a halogen such as chlorine. There is an exchange of ions resulting in the substitution of the Y⁻ ion by the BTO. This gives the TKX-50. The BTO may or may not be boiled when adding the hydroxylammonium salt. Following the addition of this salt, the TKX-50 obtained precipitates in the solution. The reaction medium can then be cooled to a temperature less than or equal to 25° C., for example comprised between 10° C. and 25° C. The reaction medium is then filtered. It is possible, if desired, to continue treating the filtrate with hydroxylammonium salt in order to increase the yield of formation of TKX-50.

Overall, over the entire synthesis, the yield of formation of TKX-50 may be greater than or equal to 80%, for example greater than or equal to 85%.

The synthesis presented is a sequential one-pot synthesis during which, at least for the azidization, cyclization, hydrolysis and ion exchange steps, a solvent is used which allows to dissolve the very sensitive reagents and reaction intermediates, and during which no isolation of the reaction intermediates which remain in solution is carried out. All the synthesis and these steps is carried out in the same reactor. Azidization, cyclization, hydrolysis and ion exchange can be carried out in a common solvent defining a reaction volume of at least 1 liter, for example at least 2 liters. The synthesis can result in obtaining a mass of TKX-50 at least equal to 500 grams, for example at least equal to one kilogram, or even several kilograms. In particular, no solvent removal is carried out between these steps. The solvent can also advantageously be selected so that the TKX-50 is insoluble in the latter and precipitates naturally once formed. The solvent can be selected from: C₁ to C₄ alcohols, for example C₁ or C₂ alcohols such as methanol or ethanol, dimethylformamide, acetone, acetonitrile, or a mixture of these solvents. In particular, it is possible to use ethanol, acetone, acetonitrile or dimethylformamide or even a mixture of acetone and dimethylformamide. Advantageously, in the synthesis of TKX-50, the use of a flammable solvent, such as diethyl ether, is dispensed with.

As indicated above, the sequential one-pot synthesis may additionally comprise, before the azidization, the formation of dichloroglyoxime by chlorination carried out by reaction between glyoxime (C₂H₄N₂O₂) and a chlorinating agent, as non-limiting example NCS (C₄H₄ClNO₂).

The chemical structure of glyoxime is provided below.

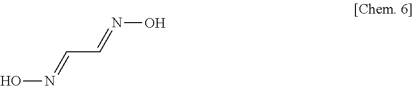

The chemical structure of NCS is provided below.

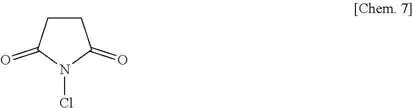

Regardless of the chlorinating agent used, a temperature comprised between 30° C. and 80° C., for example between 60° C. and 80° C., can be imposed during the chlorination. The duration of the chlorination can be comprised between 2 hours and 6 hours. As indicated above, the use of DMF as solvent is particularly advantageous in this context, allowing to carry out the sequential one-pot synthesis from the chlorination of glyoxime by NCS until finally obtaining TKX-50.

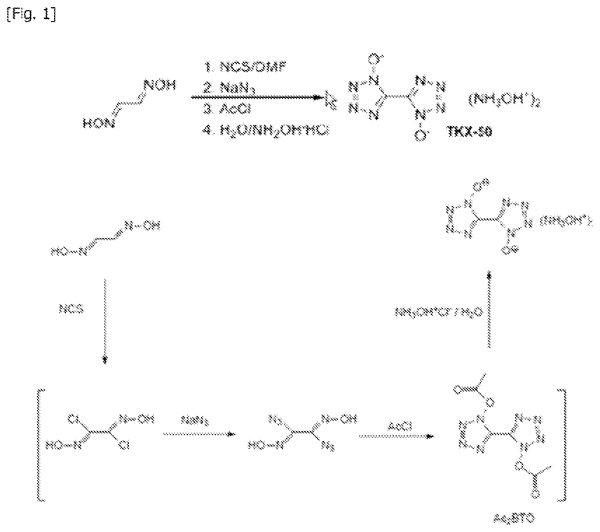

FIG. 1 provides an overall reaction diagram of an example of synthesis according to the invention using dichloroglyoxime, obtained beforehand by chlorination of glyoxime with NCS. This synthesis can be carried out entirely in dimethylformamide.

The TKX-50 can then be formulated in an energetic composition, which is for example explosive or propulsive, by techniques known per se.

### EXAMPLES

### Example 1: Sequential One-Pot Synthesis of TKX-50 from Glyoxime (According to the Invention)

N-chlorosuccinimide (NCS, 90.0 g, 674 mmol, 2.0 eq.) was added to a solution of glyoxime (20.0 g, 341 mmol) in dimethylformamide (DMF, 375 mL). The mixture was left to stir for 4 hours at 75° C. The solution was then cooled to a temperature comprised between 0° C. and 5° C. and NaN₃ (48 g, 674 mmol, 2.0 eq.) was added in portions. The reaction mixture was then stirred at this temperature for 60 minutes.

The reaction mixture was heated to 50° C. and acetyl chloride AcCl (200 mL or 150 mL) was then added and a plateau at 50° C. was imposed overnight (13 hours). The mixture was then cooled by adding ice water. After the foam disappeared, ice water was added again until a solution was obtained (total volume: 1.6 L).

The solution was heated to boiling point and hydroxylammonium chloride (60 g, 1.16 mol, 2.5 eq.) was added. The reaction medium was cooled to 20° C. The precipitate was vacuum filtered.

36.3 g (152 mmol) of TKX-50 were obtained.

The filtrate was concentrated under vacuum and reheated to 90° C. Additional hydroxylammonium chloride (5.0 g, 71 mmol) was added and the mixture cooled to 20° C. The precipitate was collected by filtration. After two days of air drying, 68.7 g (290 mmol, 85%) of crystallized TKX-50 could be obtained.

The TKX-50 obtained was characterized. The results below were obtained.

¹H NMR (400 MHz, DMSO-d₆): δ=9.70 ppm; ¹³C NMR (101 MHz, DMSO): δ=135.0 ppm.

Elemental analysis (found/calculated): C (10.65/10.47), H (3.49/3.41), N (58.56/59.31).

### Example 2: Sequential One-Pot Synthesis of TKX-50 from Dichloroglyoxime (According to the Invention)

This example shows the possibility of synthesizing TKX-50 by a sequential one-pot reaction starting from dichloroglyoxime in solvents other than DMF.

The dichloroglyoxime (2.0 g, 12.8 mmol) was dissolved in the selected solvent (ethanol, acetone, acetone/DMF 1:1 mixture or acetonitrile) (100 mL) and the solution was cooled to 0° C. Sodium azide NaN₃ (2.15 g, 32.9 mmol) was added in portions and the reaction mixture stirred at a temperature comprised between 0° C. and 5° C. for 60 minutes.

Acetyl chloride (10 mL) was then added at 50° C. The reaction medium was heated overnight (13 hours) at 50° C. then poured into ice water and heated until a solution was obtained.

Then, hydroxylammonium chloride (5.0 g, 71 mmol, 5.6 eq) was added. The reaction medium was left to stand until crystallization of the TKX-50.

The TKX-50, synthesized (with the yields indicated below) in the different solvents, was characterized by NMR:
EtOH (yield: 1.12 g, 4.74 mmol, 41%):
   ¹H NMR (400 MHz, DMSO-d₆): δ=9.80 ppm,
   ¹³C NMR (101 MHz, DMSO): δ=135.0 ppm.
Acetone 100 mL (yield: 1.93 g, 8.2 mmol, 65%):
   ¹H NMR (400 MHz, DMSO-d₆): δ=9.73 ppm,
   ¹³C NMR (101 MHz, DMSO): δ=135.0 ppm.
DMF/Acetone (yield: 2.4 g, 9.9 mmol, 78%):
   ¹H NMR (400 MHz, DMSO-d₆): δ=9.74 ppm;
   ¹³C NMR (101 MHz, DMSO): δ=135.0 ppm;
Acetonitrile (yield: 2.1 g, 8.9 mmol, 67%):
   ¹H NMR (400 MHz, DMSO-d₆): δ=9.74 ppm;
   ¹³C NMR (101 MHz, DMSO): δ=135.0 ppm;

### Example 3: Sequential One-Pot Synthesis of TKX-50 on a Larger Scale (10 L Reactor) (According to the Invention)

This example shows the possibility of synthesizing TKX-50 by a sequential one-pot reaction from glyoxime in DMF on a larger scale.

N-chlorosuccinimide (NCS, 1220 g, 9.13 mol, 2.0 eq.) was added to a solution of glyoxime (400 g, 4.54 mol) in dimethylformamide (DMF, 3.1 L) in a 10 L reactor. The mixture was stirred for 4 hours at 75° C. The solution was then cooled to a temperature comprised between 0° C. and 5° C. and NaN₃ (640 g, 9.84 mol) was added in portions. The reaction mixture was then stirred at this temperature for 60 minutes.

Acetyl chloride AcCl (0.82 L or 820 mL, 10.45 mol) was then added gradually at 50° C. The reaction mixture was then stirred overnight (13 hours) at 50° C. The mixture was then cooled by adding ice water until a solution was obtained (total volume: 3.3 L).

The solution was heated to 100° C. and hydroxylammonium chloride (948 g, 13.64 mol) was added. The reaction medium was cooled to 20° C. The precipitate was vacuum filtered.

926.65 g (3.92 mol) of TKX-50 were obtained, which corresponds to a yield of approximately 86%.

TKX-50 was characterized by NMR:

¹H NMR (400 MHz, DMSO-d₆): δ=9.73 ppm; ¹³C NMR (101 MHz, DMSO): δ=134.4 ppm.

The expression “comprised between . . . and . . . ” must be understood as including the limits.

1.-10. (canceled)

11. A method for sequential one-pot synthesis of TKX-50, comprising at least:
an azidization of a dihalogenoglyoxime or of the diaminoglyoxime so as to obtain the diazidoglyoxime,
a cyclization by reaction of the diazidoglyoxime obtained with an acetyl halide so as to obtain 1,1′-diacetyl-5,5′-bistetrazole, a temperature greater than or equal to 30° C. and less than the boiling temperature of the acetyl halide being imposed during the cyclization,
a hydrolysis of the 1,1′-diacetyl-5,5′-bistetrazole obtained into 5,5′-bistetrazole-1,1′-diolate, and
an ion exchange by adding a hydroxylammonium salt to the 5,5′-bistetrazole-1,1′-diolate obtained so as to obtain TKX-50,
a solvent allowing to dissolve the reagents and reaction intermediates being used during the synthesis, the reaction intermediates remaining in solution and no isolation of the latter being carried out, the azidization, the cyclization, the hydrolysis and the ion exchange being carried out in a common solvent comprising at least dimethylformamide, acetone or acetonitrile.

12. The method according to claim 11, wherein the common solvent comprises dimethylformamide.

13. The method according to claim 11, wherein the diazidoglyoxime is obtained by azidization of a dihalogenoglyoxime.

14. The method according to claim 13, wherein the dihalogenoglyoxime is dichloroglyoxime, and wherein the method further comprises, prior to the azidization, the formation of the dichloroglyoxime by chlorination of the glyoxime by reaction with N-chlorosuccinimide.

15. The method according to claim 14, wherein a temperature comprised between 30° C. and 80° C. is imposed during the chlorination.

16. The method according to claim 14, wherein the chlorination as well as the azidization, the cyclization, the hydrolysis and the ion exchange are carried out in dimethylformamide.

17. The method according to claim 11, wherein the diazidoglyoxime is obtained by azidization of the diaminoglyoxime.

18. A method for the manufacture of an energetic composition, comprising at least:
implementing a method according to claim 11 so as to obtain TKX-50, and
obtaining the energy composition from the TKX-50 thus obtained.
